(19) United States
(12) Patent Application Publication
(10) Pub. No.: US 2005/0215910 A1
(43) Pub. Date: Sep. 29, 2005

(54) REMOTE CONTROL CAPABLE OF MEASURING BODY TEMPERATURE

(76) Inventors: Wen-Jung Yang, Taipei City (TW); Chang-Szu Tsai, Taipei City (TW)

Correspondence Address: NORTH AMERICA INTERNATIONAL PATENT OFFICE (NAIPC), P.O. BOX 506, MERRIFIELD, VA, 22116, US

(21) Appl. No.: 10/708,754

(22) Filed: Mar. 23, 2004

(51) Int. Cl. A61B 6/00; B60Q 1/00; A61B 5/02; A61B 5/00

(52) U.S. Cl. 600/474; 600/500

(57) ABSTRACT

A remote control for controlling an electronic device includes a housing, a remote control module installed inside the housing for controlling the electronic device according to an input signal, and a body temperature measuring module installed inside the housing, for sensing body temperature by infrared rays.

### BACKGROUND OF INVENTION

1. Field of the Invention

The invention relates to a remote control of an electronic product, and more particularly, to a remote control capable of measuring body temperature.

2. Description of the Prior Art

A remote control has become an indispensable device for a user to control an electronic product from a remote distance. Please refer to FIG. 1 showing a block diagram of a conventional remote control 2. The remote control 2 includes an infrared light-emitting diode (LED) 4 for generating infrared rays, an input signal receiving module 6 for receiving input signals, and a control module 8 electrically connected between the LED 4 and the input signal receiving module 6. The control module 8 generates infrared control signals similar to Morse code according to the input signals received by the input signal receiving module 6 in order to control the LED 4 and further control an electronic device.

However, the remote control provides nothing more than remote control. It will be very useful if a daily necessity like the remote control is multifunctional.

### SUMMARY OF INVENTION

It is therefore a primary objective of the claimed invention to provide a remote control capable of measuring body temperature, in order to solve the problem mentioned above.

Briefly, a remote control for controlling an electronic device includes a housing, a remote control module installed inside the housing for controlling the electronic device according to an input signal, and a body temperature measuring module installed inside the housing for sensing body temperature by infrared rays.

These and other objectives of the present invention will no doubt become obvious to those of ordinary skill in the art after reading the following detailed description of the preferred embodiment that is illustrated in the various figures and drawings.

### BRIEF DESCRIPTION OF DRAWINGS

FIG. 1 is a block diagram of a conventional remote control.

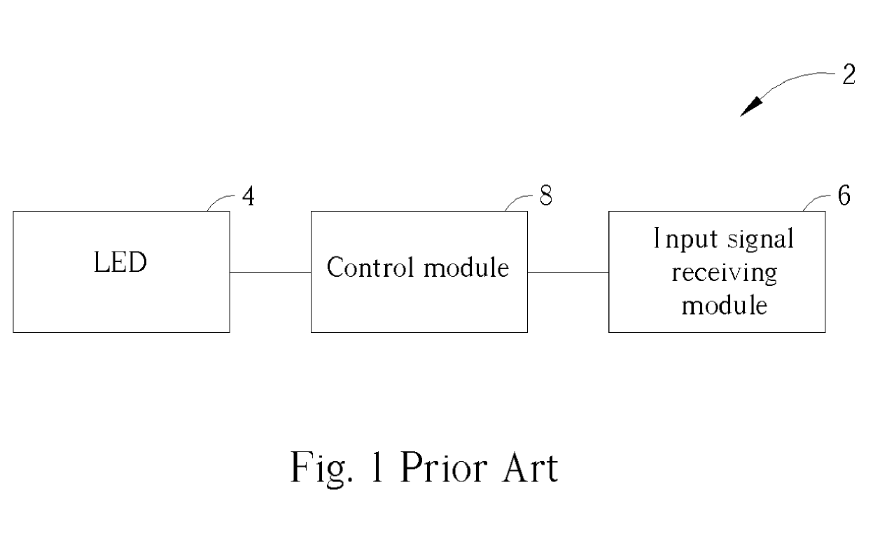

FIG. 2 is a block diagram of a remote control for controlling an electronic device according to the present invention.

FIG. 3 is an external view of the remote control according to the present invention.

FIG. 4 is a top view of the remote control.

FIG. 5 illustrates how to measure body temperature from ones forehead using the remote control.

### DETAILED DESCRIPTION

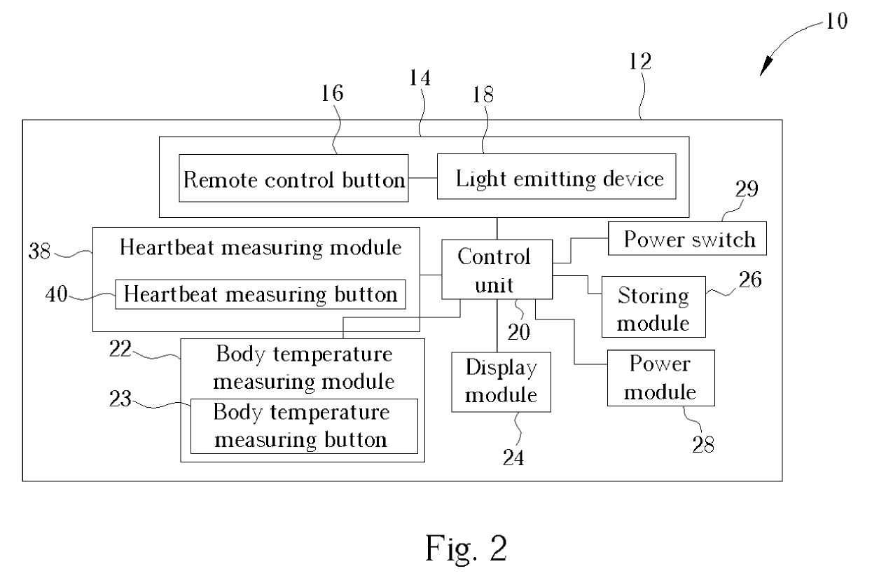

Please refer to FIG. 2 showing a block diagram of a remote control 10 for controlling an electronic device according to the present invention. The remote control 10 includes a housing 12 and a remote control module 14 installed inside the housing 12 for controlling the remote control 10 and controlling the electronic device according to an input signal. The remote control module 14 includes a plurality of remote control buttons 16 for inputting the input signal, a light emitting device 18, e.g. an infrared LED, for emitting infrared rays to control the electronic device, and a control unit 20 electrically connected to the remote control buttons 16 and the light emitting device 18 for controlling the light emitting device 18 to emit infrared rays, according to the input signal input by pressing the remote control buttons 16.

The remote control 10 further includes a body temperature measuring module 22, for example an infrared sensor for sensing body temperature by infrared rays, installed inside the housing 12. The body temperature measuring module 22 includes a body temperature measuring button 23 for activating the body temperature measuring module 22 when triggered. The remote control 10 further includes a heartbeat measuring module 38 installed inside the housing 12. A user can put a finger on a sensor 39 of the heartbeat measuring module 38 to measure heartbeat. The heartbeat measuring module 38 includes a heartbeat measuring button 40 for activating the heartbeat measuring module 38 when triggered. The remote control 10 further includes a display module 24, for example a liquid crystal display (LCD), for displaying the body temperature measured by the body temperature measuring module 22 and the heartbeat measured by the heartbeat measuring module 38, a storing module 26 for storing the body temperature measured by the body temperature measuring module 22 and the heartbeat measured by the heartbeat measuring module 38, a power module 28, e.g. a battery, for providing power to the remote control 10, and a power switch 29 for turning on/off the remote control 10.

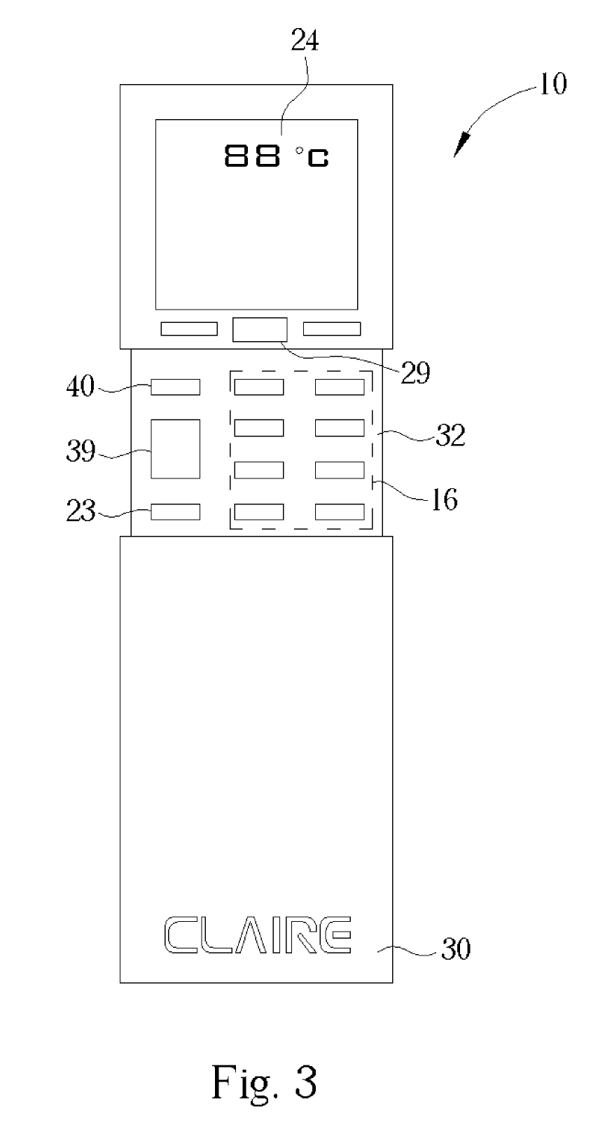

Please refer to FIG. 3 showing an external view of the remote control 10 according to the present invention. The housing 12 of the remote control 10 includes a movable cover 30 covering a main housing 32 in order to prevent dust from settling on the remote control buttons 16 on the main housing 32. When using the remote control 10, drag the cover 30 and press the remote control buttons 16 to input the input signals corresponding to different functions. For instance, if the remote control 10 is for an air conditioner, the remote control buttons 16 relates to the timer, temperature, etc. The user can read related information of the electronic device from the display module 24 on the main housing 32. The housing 12 further includes the power switch 29 for turning on/off the remote control 10, the body temperature measuring button 23, and the heartbeat measuring button 40. When the user presses the body temperature measuring button 23, the body temperature measuring module 22 will then start to measure body temperature. And when the user presses the heartbeat measuring button 40 and put a finger on the sensor 39, the heartbeat measuring module 38 will then start to measure heartbeat.

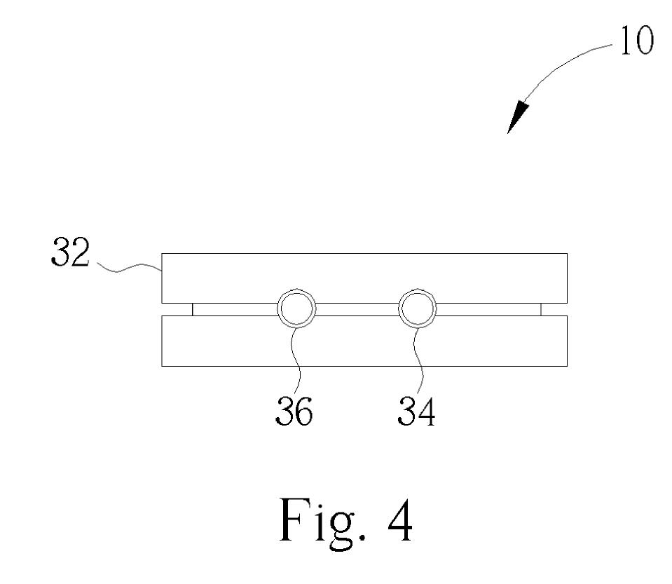

Please refer to FIG. 4 showing a top view of the remote control 10. The main housing 32 has a first aperture 34 and a second aperture 36. The light emitting device 18 of the remote control module 14 emits infrared rays toward the first aperture 34, and the body temperature measuring module 22 scans infrared rays emitted from human body to sense body temperature through the second aperture 36.

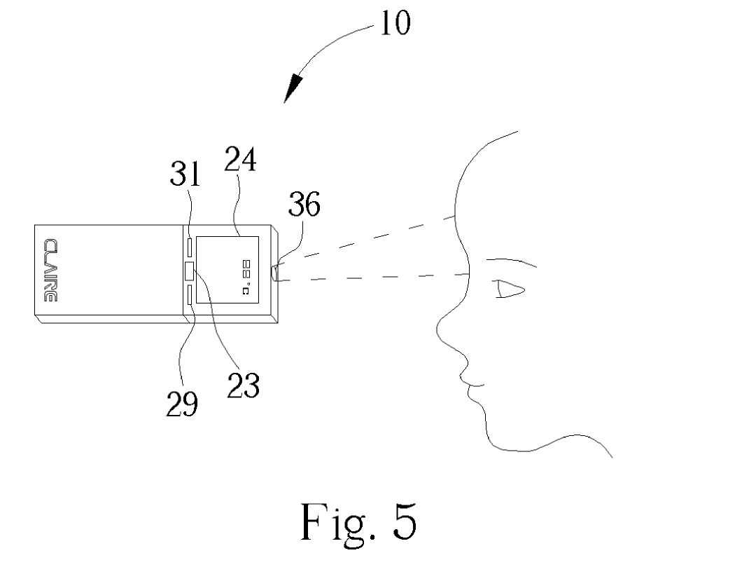

Please refer to FIG. 5 showing how to measure body temperature from ones forehead using the remote control 10. The user presses the body temperature measuring button 23. Afterwards, the user puts the second aperture 36 toward the forehead of an examinee so that the body temperature measuring module 22 scans infrared rays emitted from a human body through the second aperture 36 to sense the body temperature. The body temperature sensed by the body temperature measuring module 22 is then transmitted to the control module 20 to be stored in the storing module 26 for a reference to the user or to be displayed by the display module 24.

The remote control 10 can also control the electronic device according to the body temperature sensed by the body temperature measuring module 22. For instance, if the remote control 10 is for an air conditioner, after the user measures his body temperature, the body temperature measuring module 22 transmits body temperature information to the control module 20 so that the control module 20 determines an optimal environment parameter, e.g. optimal temperature, humidity or wind direction, according to the information. In such a manner, a parameter corresponding to the user is considered so that the air conditioner can be more ergonomic.

In contrast to the prior art, the present invention provides the remote control capable of measuring body temperature and heartbeat as well as controlling the electronic device. Moreover, the remote control is capable of controlling the electronic device ergonomically according to a sensed body temperature.

Those skilled in the art will readily observe that numerous modifications and alterations of the device may be made while retaining the teachings of the invention. Accordingly, the above disclosure should be construed as limited only by the metes and bounds of the appended claims.

1. A remote control for controlling an electronic device comprising:
a housing;
a remote control module installed inside the housing for controlling the electronic device according to an input signal; and
a body temperature measuring module installed inside the housing for sensing body temperature by infrared rays.

2. The remote control of claim 1 further comprising a display module for displaying the body temperature measured by the body temperature measuring module.

3. The remote control of claim 2 wherein the display module is a liquid crystal display (LCD).

4. The remote control of claim 1 further comprising a storing module for storing the body temperature measured by the body temperature measuring module.

5. The remote control of claim 1 wherein the remote control module comprises a plurality of remote control buttons for inputting the input signal, and a light emitting device for emitting infrared rays for controlling the electronic device, and the remote control further comprises a control unit electrically connected to the plurality of buttons and the light emitting device for controlling the light emitting device to emit the infrared rays according to the input signal.

6. The remote control of claim 5 wherein the control unit receives the body temperature information from the body temperature measuring module and controls the electronic device according to the body temperature information.

7. The remote control of claim 5 wherein the light emitting device of the remote control module is an infrared light emitting diode (LED).

8. The remote control of claim 1 wherein the body temperature measuring module is an infrared sensor.

9. The remote control of claim 1 further comprising a heartbeat measuring module for measuring heartbeat.
